(19) United States
(12) Patent Application Publication
(10) Pub. No.: US 2025/0246282 A1
(43) Pub. Date: Jul. 31, 2025

(54) DIGITAL THERAPY MANAGEMENT SYSTEM OF REFLECTING AND UPDATING FEEDBACK ACCORDING TO ACTUAL USE OF DIGITAL THERAPEUTICS AND OPERATION METHOD THEREOF

(71) Applicant: ODN CO., Ltd., Seoul (KR)

(72) Inventor: Sang Youl RHEE, Seoul (KR)

(73) Assignee: ODN CO., Ltd., Seoul (KR)

(21) Appl. No.: 18/922,987

(22) Filed: Oct. 22, 2024

(30) Foreign Application Priority Data
Jan. 31, 2024 (KR) 10-2024-0014816

(51) Int. Cl. G16H 20/00; G16H 10/60; G16H 40/20; G16H 40/40; G16H 40/67

(52) U.S. Cl. CPC G16H 20/00; G16H 10/60; G16H 40/20; G16H 40/40; G16H 40/67

(57) ABSTRACT

Disclosed are a digital therapy management system and method. More particularly, a technical idea of a digital therapeutic platform configured to collect information accumulated due to patient's intentional/unintentional digital therapeutics use and reflect and update feedback according to actual use of the digital therapeutics in a process of prescribing and managing a digital therapeutics in conjunction with a medical institution is provided.

The digital therapy management system according to an embodiment may include an implementation result manager for collecting implementation result information generated according to actual use of one or more digital therapeutics based on prescription information generated from a terminal of a medical institution; an analyzer/evaluator for analyzing and evaluating the collected implementation result information; a feedback information generator for generating feedback information based on the analyzed and evaluated result; and a feedback information provider for providing the generated feedback information to a corresponding system to adjust an input-side system environment required to generate the digital therapeutics or an output-side system environment according to the actual use of the digital therapeutics.

This work was supported by the Ministry of SMEs and Startups (MSS) under the Technology Innovation Program (TIPS) funded by the Korean government (Grant No. RS-2023-00303873, ICT-based personalized metabolic disease management program (digital therapeutics)).

### CROSS-REFERENCE TO RELATED APPLICATION

This application claims priority to Korea Patent Application No. 10-2024-0014816, filed on Jan. 31, 2024 in the Korea Intellectual Property Office, the disclosure of which is incorporated herein by reference.

This work was supported by the Ministry of SMEs and Startups (MSS) under the Technology Innovation Program (TIPS) funded by the Korean government (Grant No. RS-2023-00303873, ICT-based personalized metabolic disease management program (digital therapeutics)).

### BACKGROUND OF THE DISCLOSURE

### Field of the Disclosure

The present disclosure relates to a digital therapy management system and an operation method thereof, and more particularly to a technical idea of a digital therapeutic platform configured collect to information accumulated due to patient's intentional/unintentional digital therapeutics use and reflect and update feedback according to actual use of the digital therapeutics in a process of prescribing and managing a digital therapeutic device (hereinafter referred to as “digital therapeutics”) in conjunction with a medical institution.

### Description of the Related Art

Recently, a digital therapeutic device (hereinafter referred to as “digital therapeutics”) is rapidly emerging as an alternative to address the unmet needs due to the increase in chronic diseases caused by the aging population, the resulting increased social burden on health insurance finances, and the problem of medical accessibility.

Digital therapeutics can be defined as software medical devices for providing evidence-based therapeutic interventions to patients to prevent, manage, and treat medical disorders or diseases. These digital therapeutics have the advantage of having less toxicity and side effects than existing treatments due to the nature of software, and can be supplied in large quantities at low cost because they do not require manufacturing, transportation, or storage necessary in general drugs, which can lower medical costs. In addition, a small number of doctors can manage a large number of patients regardless of physical and time limitations, which can partially compensate for problems such as health insurance finances, lack of medical supply, and regional bias.

As these digital therapeutics are expected to play a key role in future health management services, the related needs are steadily increasing. However, existing systems have a problem in that the prescribing and managing of digital therapeutics is inefficient due to insufficient connection with existing medical systems and users.

### Related Art Documents

### Patent Documents

(Patent Document 1) Korea Patent Application Publication No. 10-2022-0034806, entitled “ADAPTIVE INTERVENTIONS FOR GASTROINTESTINAL HEALTH CONDITIONS”
(Patent Document 2) Korea Patent Application Publication No. 10-2023-0149384, entitled “SYSTEM AND METHOD FOR EVALUATING USABILITY OF DIGITAL THERAPEUTIC AGENT AND MEDICAL DEVICE USING METAVERSE”
(Patent Document 3) Korea Patent No. 2570479, entitled “DIGITAL THERAPEUTICS PLATFORM SYSTEM AND METHOD APPLYING SELECTIVE DE- IDENTIFICATION OF SENSITIVE INFORMATION BASED ON ARTIFICIAL INTELLIGENCE”

### SUMMARY OF THE DISCLOSURE

Therefore, the present disclosure has been made in view of the above problems, and it is an object of the present disclosure to provide a digital therapy management system and method capable of providing a high-quality digital therapeutic optimized for a user by linking various health information on the user with medical records of a medical institution.

It is another object of the present disclosure to provide a digital therapy management system and method capable of diagnosing treatment results more accurately based on user's feedback on prescribed digital therapeutics.

It is yet another object of the present disclosure to provide a digital therapy management system linked to an existing medical record database to more easily manage user's medical records related to digital therapeutics with only an update operation, and its method. In accordance with an aspect of the present disclosure, the above and other objects can be accomplished by the provision of a digital therapy management system, including: an implementation result manager for collecting implementation result information generated according to actual use of one or more digital therapeutics based on prescription information generated from a terminal of a medical institution; an analyzer/evaluator for analyzing and evaluating the collected implementation result information; a feedback information generator for generating feedback information based on the analyzed and evaluated result; and a feedback information provider for providing the generated feedback information to a corresponding system to adjust an input-side system environment required to generate the digital therapeutics or an output-side system environment according to the actual use of the digital therapeutics.

The feedback information provider according to an embodiment may provide feedback information for adjusting settings for at least one device among a multimodal sensor, VR/AR equipment, or wearable device for collecting user information as an input-side system environment necessary for generating the digital therapeutics.

The feedback information provider according to an embodiment may provide feedback information processing one or more data of questionnaire data, medical knowledge, and guidelines provided to a user as an input-side system environment necessary for generating the digital therapeutics.

The feedback information provider according to an embodiment may provide the generated feedback information to a corresponding system to adjust an output-side system environment for a service provided by a physical hospital or virtual hospital that supports the actual use of the digital therapeutics.

The feedback information provider according to an embodiment may provide the generated feedback information to a corresponding system so as to control the output-side system environment that provides one or more services among digital literacy service, evidence-based/personalized service, and virtual hospital medical service as a service provided by a physical hospital or a virtual hospital.

The implementation result manager according to an embodiment may collect at least one of data, input by a user, and bio-signal data of the user as the implementation result information in response to the provided prescription information, the data input by the user may include at least one of the number of times of implementation, implementation time, and implementation intensity of digital therapeutics corresponding to the prescription information, and the bio-signal data of the user may be monitored through at least one of a smart terminal and IoT terminal of the user and may include at least one of blood pressure, blood sugar, body temperature, heart rate, moving distance, and moving steps of the user.

The implementation result manager according to an embodiment may provide the prescription information to a user's terminal including a multimodal sensor, may receive implementation result information corresponding to the provided prescription information from the user's terminal, and may provide the received implementation result information to the terminal of the medical institution.

The digital therapy management system according to an embodiment may further include a record manager configured to receive treatment result information corresponding to the implementation result information from the terminal of the medical institution and to update the received treatment result information in electronic medical record (EMR) database of the medical institution, wherein the treatment result information includes at least one information of the prescription information, the implementation record information, medical professional's comment information according to the implementation record information and the medical professional's authentication information.

In accordance with another aspect of the present disclosure, provided is an operation method of a digital therapy management system, the operation method including: collecting implementation result information generated according to actual use of one or more digital therapeutics based on prescription information generated from a terminal of a medical institution; analyzing and evaluating the collected implementation result information; generating feedback information based on the analyzed and evaluated result; and providing the generated feedback information to a corresponding system to adjust an input-side system environment required to generate the digital therapeutics or an output-side system environment according to the actual use of the digital therapeutics.

The providing of the generated feedback information to a corresponding system according to an embodiment may include providing feedback information for adjusting settings for at least one device among a multimodal sensor, VR/AR equipment, or wearable device for collecting user information as an input-side system environment necessary for generating the digital therapeutics.

The providing of the generated feedback information to a corresponding system according to an embodiment may include providing feedback information processing one or more data of questionnaire data, medical knowledge, and guidelines provided to a user as an input-side system environment necessary for generating the digital therapeutics.

The providing of the generated feedback information to a corresponding system according to an embodiment may include providing the generated feedback information to a corresponding system to adjust an output-side system environment for a service provided by a physical hospital or virtual hospital that supports the actual use of the digital therapeutics.

The providing of the generated feedback information to a corresponding system according to an embodiment may include providing the generated feedback information to a corresponding system so as to control the output-side system environment that provides one or more services among digital literacy service, evidence-based/personalized service, and virtual hospital medical service as a service provided by a physical hospital or a virtual hospital.

### BRIEF DESCRIPTION OF THE DRAWINGS

The above and other objects, features and other advantages of the present disclosure will be more clearly understood from the following detailed description taken in conjunction with the accompanying drawings, in which:

FIG. 1 illustrates a digital therapy management system according to an embodiment;

FIG. 2 is a drawing explaining a meta-hospital system architecture to which the digital therapy management system according to an embodiment is applied;

FIG. 3 is a diagram illustrating a UX evaluation process through UX user measurement and UX expert evaluation and analysis;

FIG. 4 is a drawing specifically explaining a UX user measurement system;

FIG. 5 is a drawing specifically illustrating a UX expert evaluation and analysis system;

FIG. 6 illustrates a drawing explaining UX merge and UX evaluation analysis through measurement metrics; and

FIG. 7 is a drawing explaining an operation method of the digital therapy management system according to an embodiment.

### DETAILED DESCRIPTION OF THE DISCLOSURE

Specific structural and functional descriptions of embodiments according to the concept of the present disclosure disclosed herein are merely illustrative for the purpose of explaining the embodiments according to the concept of the present disclosure. Furthermore, the embodiments according to the concept of the present disclosure can be implemented in various forms and the present disclosure is not limited to the embodiments described herein.

The embodiments according to the concept of the present disclosure may be implemented in various forms as various modifications may be made. The embodiments will be described in detail herein with reference to the drawings. However, it should be understood that the present disclosure is not limited to the embodiments according to the concept of the present disclosure, but includes changes, equivalents, or alternatives falling within the spirit and scope of the present disclosure.

The terms such as “first” and “second” are used herein merely to describe a variety of constituent elements, but the constituent elements are not limited by the terms. The terms are used only for the purpose of distinguishing one constituent element from another constituent element. For example, a first element may be termed a second element and a second element may be termed a first element without departing from the scope of rights according to the concept of the present disclosure.

It will be understood that when an element is referred to as being “on”, “connected to” or “coupled to” another element, it may be directly on, connected or coupled to the other element or intervening elements may be present. In contrast, when an element is referred to as being “directly on,” “directly connected to” or “directly coupled to” another element or layer, there are no intervening elements or layers present. Other words used to describe the relationship between elements should be interpreted in a like fashion (e.g., “between,” versus “directly between,” “adjacent,” versus “directly adjacent,” etc.).

The terms used in the present specification are used to explain a specific exemplary embodiment and not to limit the present inventive concept. Thus, the expression of singularity in the present specification includes the expression of plurality unless clearly specified otherwise in context. Also, terms such as “include” or “comprise” in the specification should be construed as denoting that a certain characteristic, number, step, operation, constituent element, component or a combination thereof exists and not as excluding the existence of or a possibility of an addition of one or more other characteristics, numbers, steps, operations, constituent elements, components or combinations thereof.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The present disclosure will now be described more fully with reference to the accompanying drawings, in which exemplary embodiments of the disclosure are shown. This disclosure may, however, be embodied in many different forms and should not be construed as limited to the exemplary embodiments set forth herein. Like reference numerals in the drawings denote like elements.

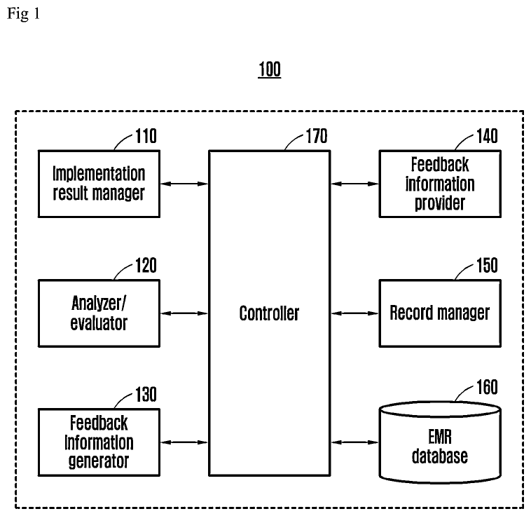

FIG. 1 illustrates a digital therapy management system according to an embodiment.

The digital therapy management system according to an embodiment collects information accumulated due to patient's intentional/unintentional DTx use.

It may collect actual users' compliance rate, usage frequency, pattern, usage time, etc. for an original prescription plan, or collect subjective feelings and satisfaction while using it.

In addition, the digital therapy management system according to an embodiment may collect key information about the clinical changes of patients regarding the use of DTx.

For example, changes in vital signs, changes in test results, changes in drug use, and clinical events (complications, emergencies, deaths, etc.) may be collected, and the collected information may be linked, and then provided to a developer/manager.

It may be utilized when DTx needs to be updated in terms of UI/UX, or when an update is needed in a DTx-hospital system linkage system.

The digital therapy management system according to an embodiment may recommend an optimal DTx that considers various clinical information such as an individual's age, gender, underlying disease, and medication information by linking the previously collected information, or may provide a patient or medical staff with information on DTx-linked clinical decision-making methodology updates.

More specifically, referring to FIG. 1, the digital therapy management system (i.e., the digital therapeutic platform or DTx platform) 100 according to an embodiment may provide a high-quality digital therapeutic optimized for a user by linking various health information on the user with medical records of a medical institution.

In addition, the digital therapy management system 100 may diagnose treatment results more accurately based on user's feedback on prescribed digital therapeutics.

In addition, the digital therapy management system 100 may be linked to an existing medical record database to more easily manage user's medical records related to digital therapeutics with only an update operation.

For this, the digital therapy management system 100 may include an implementation result manager 110, an analyzer/evaluator 120, a feedback information generator 130, a feedback information provider 140, a record manager 150, an EMR database 160, and a controller 170.

The implementation result manager 110 may collect implementation result information generated according to the actual use of at least one of digital therapeutics based on prescription information generated from a terminal of a medical institution.

The digital therapy management system 100 monitors digital therapeutics prescribed to patients by medical professionals through the implementation result manager 110 and collects data on patients using the therapeutics. Through this data collection, the system may obtain various information related to the effectiveness and side effects of the digital therapeutics and patient response thereto. This may help medical professionals optimize treatment plans and provide personalized treatment to patients. In addition, the collected information may be utilized in research and development activities to contribute to improving the efficacy of digital therapeutics and leading to better treatment results.

The implementation result manager 110 may collect at least one of data, input by a user, and user's bio-signal data in response to provided prescription information as the implementation result information, the data input by the user may include at least one of the number of times of implementation, implementation time, and implementation intensity of the digital therapeutics corresponding to the prescription information, and the user's bio-signal data may be monitored through at least one of a smart terminal and IoT terminal of the user and may include at least one of blood pressure, blood sugar, body temperature, heart rate, moving distance, and moving steps of the user.

The implementation result manager 110 may provide the prescription information to a user's terminal including a multimodal sensor, receive the implementation result information corresponding to the provided prescription information from the user's terminal, and provide the received implementation result information to the terminal of the medical institution.

The implementation result manager 110 may integrate a multimodal sensor into the user's terminal to collect various information and transmit the prescription information of the digital treatment provided to the user. The user's terminal performs digital treatment according to the prescription information and generates various bio-signals and user input data that occur. These data may be used to comprehensively understand user's physical activity, physiological response, and behavioral patterns by utilizing a multimodal sensor.

The implementation result manager 110 may analyze the various data received from the user's terminal and track the effect, side effects, and individual responses of the corresponding digital treatment in real-time. This information may be transmitted to a terminal of a medical institution, contributing to medical professionals' in-depth understanding of the patient's condition and optimizing individual treatment plans.

In addition, the use of the multimodal sensor may enable a more comprehensive assessment of the user's health status, and provision of information on environmental factors in addition to bio-signals. This provides healthcare professionals with a richer context to help develop patient-centered treatment approaches and ultimately improve patient health and well-being.

The analyzer/evaluator 120 may analyze and evaluate the collected implementation result information.

The analyzer/evaluator 120 may utilize advanced data analysis and evaluation technologies in this process to further understand and optimize the effectiveness of digital treatment and the user's response.

The analyzer/evaluator 120 may identify various data patterns, trends, and statistical indicators to track the performance of the digital treatment. For example, it may be confirmed whether the user's bio-signal pattern is effectively improved at a specific time zone, or whether a specific user group has a special effect, etc. These analysis results may be used to derive effective treatment strategies at the individual user and group level.

For example, artificial intelligence algorithms may play a key role in these analysis and evaluation processes. For example, machine learning and pattern recognition algorithms may be used to discover meaningful patterns in large amounts of data and train predictive models. This allows the prediction of the most effective digital treatment combination for a specific patient and the establishment of an individually tailored treatment plan.

In addition, artificial intelligence may help medical professionals gain insight and intervene quickly by interpreting interactions and complex relationships between data. Through this analysis and evaluation process, effective application and continuous improvement of digital treatment are possible, and patient-centered treatment methods may be implemented more precisely.

The feedback information generator 130 may generate feedback information based on the analyzed and evaluated results.

In this process, the user's performance of digital treatment may be comprehensively evaluated, and the information may be processed to provide an improved treatment experience to an input or output system.

The feedback information generator 130 may generate feedback in various forms. For example, personalized advice or motivational messages about individual treatment outcomes and areas to be improved may be provided to a user. In addition, a comprehensive report or recommendation regarding a patient's progress may be generated for medical professionals to support decision-making.

The feedback information provider 140 may provide the generated feedback information to the corresponding system to adjust an input-side system environment required to generate one or more digital therapeutics or an output-side system environment according to the actual use of the digital therapeutics.

The feedback information provider 140 uses the generated feedback information to effectively improve and optimize one or more digital therapeutics. For this, the user experience may be improved and the effectiveness of the treatment may be maximized by dynamically adjusting the input-side system environment or the output-side system environment according to the actual use of the digital therapeutics.

The feedback information provider 140 generates various forms of feedback to control the system environment. For example, feedback may be provided to a specific user to adjust the composition or intensity of individual treatment sessions. Alternatively, measures such as improving the interface of the system or activating/deactivating specific functions may be taken depending on the usage pattern of the digital therapeutic.

This feedback information provider 140 provides a customized treatment environment by adapting to user's requirements and changes in user's condition, which contributes to increasing patient participation and optimizing the effectiveness of treatment. In addition, the feedback information provider 140 may monitor the performance of the digital therapeutics and perform appropriate updates and improvements according to new research results or medical knowledge to achieve continuous evolution.

That is, the feedback information provider 140 may play a pivotal role in continuously optimizing the patient-centered digital treatment environment to improve the patient's health and well-being.

The feedback information provider 140 is an input-side system environment required to generate one or more digital therapeutics digital therapeutic, and may adjust settings for at least one of a multimodal sensor, VR/AR equipment, or wearable device for collecting user information.

For example, the feedback information provider 140 may dynamically adjust the settings of a multimodal sensor for collecting user's behavior and bio-signals. For example, the sensitivity of a sensor may be adjusted by analyzing the walking pattern of a specific user or considering the interaction with the heart rate.

In digital treatment utilizing virtual or augmented reality, the setting of VR/AR equipment is important. The feedback information provider 140 may dynamically adjust elements such as the resolution, field of view, or audio settings of the device in consideration of the user's preference or convenience.

The wearable device may be used to collect the user's bio-signals in real-time. The feedback information provider 140 may ensure accurate and effective data collection by adjusting settings such as the sensitivity, cycle, or specific sensor activation of the wearable device.

By dynamically adjusting the settings in this way, the feedback information provider 140 may provide a customized and optimized digital treatment environment to the user, and improve the efficiency of data collection to help medical professionals make better treatment decisions.

The feedback information provider 140 is an input-side system environment required to generate one or more digital therapeutics, and may provide feedback information that processes at least one data among survey data, physician knowledge, or guidelines provided to the user.

The results of a survey given to the user may be analyzed and the information may be utilized to evaluate the effectiveness of the digital treatment and generate feedback. For example, future treatment plans may be adjusted by considering the user's preference for a specific treatment method or opinions on effective changes.

Feedback information may be generated by utilizing the medical knowledge of doctors. By providing a user with a comprehensive set of professional opinions of doctors or the latest research results on treatment methods, it is possible to make better treatment decisions.

In addition, feedback information may be generated based on medical guidelines or treatment recommendations. Through this, it is possible to provide a recommended treatment method according to a user's current condition and support the user by explaining the treatment progress according to the guidelines.

The feedback information provider 140 may provide the generated feedback information to the corresponding system to adjust an output-side system environment for a service provided by a physical hospital or virtual hospital that supports the actual use of one or more digital therapeutics.

The feedback information provider 140 may dynamically adjust the settings of the corresponding system for the digital treatment service provided by the physical hospital. For example, a treatment method specialized for a specific patient group may be applied, or a digital treatment session may be adjusted to fit the operating schedule of a physical hospital.

The feedback information provider 140 may optimize the system according to the characteristics of a virtual environment for a digital treatment service in a virtual hospital. The digital treatment service of the virtual hospital may be improved by considering user interaction in the virtual environment, integration of a video consultation system, or real-time data transmission.

The feedback information provider 140 is a service provided by a physical hospital or a virtual hospital, and may provide the generated feedback information to the corresponding system to adjust an output-side system environment that provides at least one service among digital literacy service, evidence-based/personalized service, or virtual hospital medical service.

Since this feedback is generated based on data, it may provide accurate and personalized content. For example, information that adjustment or change of a specific treatment element is necessary based on a specific user's behavior pattern may be provided. In addition, this feedback information may contribute to increasing user participation and maximizing the effectiveness of treatment.

The feedback information generator 130 plays a key role in improving the effectiveness of digital treatment through continuous monitoring and analysis and providing treatment optimized for the patient's health condition.

The record manager 150 may receive treatment result information corresponding to the implementation result information from a terminal of a medical institution, and update the received treatment result information in the electronic medical record (EMR) database of the medical institution.

EMR stores patient's medical records in digital form. This may improve the safety, efficiency, and accessibility of medical records by managing medical records in electronic form instead of paper.

EMR contains various medical information such as patient diagnosis, prescription, test results, treatment plan, allergy information, and medical history. This helps to understand and manage a patient's comprehensive medical condition.

EMR allows medical professionals to access patient information in real-time, and may facilitate collaboration on patient treatment by allowing multiple medical professionals to simultaneously view EMR and share necessary information.

Electronic records may reduce medical errors. Correctly recorded information helps medical staff provide accurate and safe treatment to patients.

EMR may efficiently manage medical records and analyze data required for medical research or quality improvement.

Patients may access their EMRs to check and manage their health information. This helps to enhance patient participation and increase awareness of medical decisions.

Treatment result information may include at least one of prescription information, the above-mentioned implementation record information, the medical professional's comment information according to the above-mentioned implementation record information, and the medical professional's authentication information.

For example, the medical institution may include at least one of a primary hospital, a secondary hospital, and a tertiary hospital, the terminal of the medical institution may include at least one of a personal computer (PC) and smart terminal equipped by a medical professional (e.g., a doctor and a nurse) belonging to the medical institution, and the terminal of the user may include at least one of a PC, smart terminal, and IoT terminal equipped by the user, without being limited thereto. The terminal of the medical institution and the terminal of the user may further include a separately certified digital therapeutic device that provides functions related to digital therapeutics.

In addition, among the user's terminals, the IoT terminal may include a wearable device worn by the user, and may be linked with the digital therapy management system 100 through direct communication and indirect communication via a smart terminal.

According to an aspect, each of the terminals of the medical institution and the terminals of the user may be linked with the digital therapy management system 100 through at least one service among separately built App services and Web services for prescribing and managing digital therapeutics. Here, the service may provide at least one of a medical institution-only service and an individual user-only service.

The EMR database 160 is a database that collects and manages EMR data of a medical institution, and may be provided on a separate medical data server equipped in a medical institution.

In addition, the EMR database 160 may collect user's personal health records (PHR) and may be provided in the digital therapy management system 100 or a separate health record server.

According to an aspect, the controller 170 may provide a registration function for the terminal of the medical institution and the terminal of the user through at least one of App services and Web services.

Specifically, the controller 170 may perform an authentication procedure when receiving an authorization request signal for using the app service and/or web service from at least one terminal among terminals of a medical institution and user's terminals, and may register at least one terminal and grant usage rights based on the result of the authentication procedure.

In addition, the controller 170 may map the digital therapeutic device with at least one of an EDI code and a non-coverage code if at least one terminal that provided the authorization request signal is a digital therapeutic device.

Meanwhile, the controller 170 may search for digital therapeutics available in the digital therapy management system 100 at the request of the terminal of the registered medical institution, may register one or more digital therapeutics derived from the search result as digital therapeutics available for use in the medical institution, and may map each of registered digital therapeutics with a hospital prescription (fee) code.

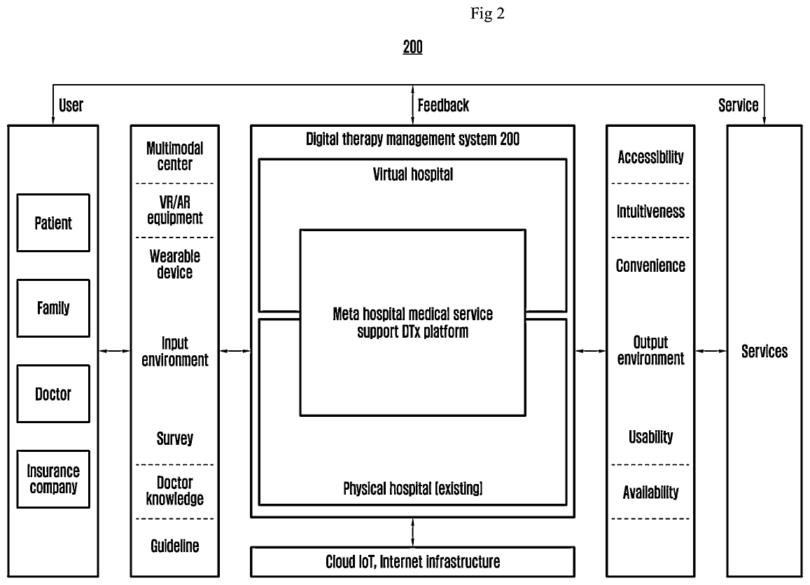

FIG. 2 is a drawing explaining a meta-hospital system architecture to which the digital therapy management system according to an embodiment is applied.

The meta-hospital system architecture applied with the digital therapy management system according to an embodiment may include an input-side system environment that collects at least one data from among multimodal sensors, VR/AR equipment, or wearable devices for collecting user information, questionnaire data provided to users, physician knowledge, or guidelines.

In addition, it may include an output-side system environment that provides at least one service of a digital literacy service, an evidence-based/personalized service, and a virtual hospital medical service as a service provided by a physical hospital or a virtual hospital.

In addition, the digital therapy management system 200 may update an input-side system environment or an output-side system environment using implementation result information generated according to the actual use of one or more digital therapeutics.

The meta-hospital system architecture applied with the digital therapy management system according to an embodiment may provide a digital therapeutic platform core module to implement a neural-symbolic AI technology, which can support the requirements of evidence-based medicine, and a personalized service support knowledge merging technology while structurally supporting virtual hospitals and physical hospitals (existing hospitals).

A situational awareness module of the meta-hospital system architecture may be largely composed of a low-level situational awareness module and a high-level situational awareness module.

In low-level situational awareness, information such as the number of steps, pulse rate, weight, and body temperature may be extracted through various sensors, and an SNS analyzer and a situation detector may be used to determine the patient's situation and current physical condition. Based on this information, in high-level situational awareness, situations (contexts) such as life patterns and lifestyles may be inferred, whether the context is correct may be verified through a situation verifier, and this context may be stored in a lifelog storage.

A medical report generation module of digital therapeutics may be composed of a visual-verbal pre-learning model, an explainable neural network model, and a neural-symbolic inference model.

In the visual-verbal pre-learning model, data is processed so that AI can learn based on image information such as patient's X-ray, CT scan, and body information.

Through these data, an explainable neural network model and a neural-symbolic inference model may learn to generate guideline-level medical reports containing patient treatment and prescription plans.

A formal knowledge acquisition module extracts text information related to the patient's MRI, BMI, blood pressure, pulse, gender, and age tabular data through a data acquisition module and transmits it to an ensemble feature selection model.

Each feature is scored and a threshold is set to structure it into a combined dataset, and the combined dataset derives a prototype from a black box model in a counterfactual generator. This prototype and the combined dataset may generate results as a data-based knowledge base through a rule generator and may be finally transferred to the knowledge merge module.

The DTx recommendation module of digital therapeutics is largely composed of a service orchestrator, a recommendation builder, and a recommendation interpreter. The service orchestrator receives symptom information data from a patient and transfers it to the recommendation builder.

The recommendation builder generates symptom information based on rules learned through AI, and the recommendation interpreter consists of tools that interpret the context or manage explainable AI, so that it may recommend rule results based on the patient's symptoms.

Due to the nature of digital therapeutics in which patients interact with the digital therapeutic service on their own and receive treatment, it is important to enable patients to use the service actively (motivated) and efficiently (easily).

For this, patient-centered UIUX optimization that can provide high usability (=efficiency) and high immersion (=activity) to patients is essential.

In general, usability enhancement and immersion enhancement are conflicting factors, so it is important to achieve a balance between the two factors according to the characteristics of each disease when implementing an actual meta-hospital.

First, the present disclosure may provide a metaverse technology as a means to specifically instruct or control user actions required for diagnosis, treatment, and aftercare services for patients using digital therapeutics.

In the case of treatment with digital therapeutics, patients interact more naturally in the immersive environment of the metaverse than in general interactions, and by inducing natural interactions during treatment, a wide range of patient information that is much closer to daily life may be obtained.

In addition, treatment guidelines may be delivered much more directly through the linkage between the treatment interface and immersive, experiential content in the metaverse.

In conclusion, it is possible to provide a metaverse environment where treatment with digital therapeutics is possible, linkage with a digital therapeutics system, a treatment protocol utilizing metaverse interaction, etc.

The present disclosure may provide a metaverse core technology for providing a realistic or hyper-realistic situation that feels like a face-to-face medical procedure between a patient and a doctor in a telemedicine communication environment.

During treatment with digital therapeutics, it is often necessary for a doctor and a patient to perform direct real-time communication for patient information, diagnosis, etc.

For this, a metaverse system of providing the same level of immersion and realism as performing communication in the same physical environment even if it is remote may be implemented.

Through this, it is possible to remotely provide a five-sensory diagnostic means that is almost the same level as a diagnosis in a physical environment.

Third, it is possible to provide the metaverse technology for supporting an environment in which patients and medical staff can mutually trust and empathize (Rapport) in telemedicine and easily understand medical practices.

In particular, it is possible to utilize the metaverse as a more efficient information delivery medium for evidence-based treatment and intervention.

A patient-centered DTx system that can convey various information that a doctor wants to convey to a patient in the form of experiential information utilizing the five senses rather than in the form of general verbal or text may be implemented.

The doctor's web and patient's app supported by the digital therapy management system according to the present disclosure should have an intuitive user interface design, be easy to understand, and be convenient to use in real-time.

As the satisfaction level of the user experience is high, the trust of users and medical staff in digital therapeutics may be high.

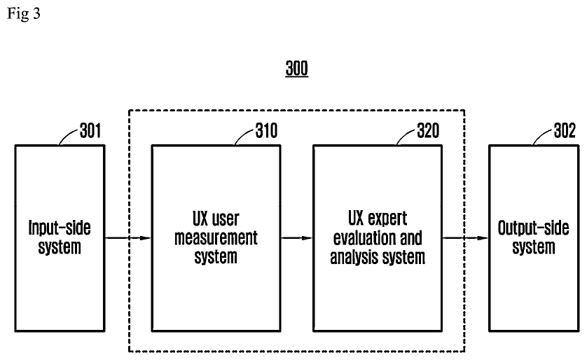

FIG. 3 is a diagram illustrating a UX evaluation process through UX user measurement and UX expert evaluation and analysis.

A digital therapy management system 300 collects data from an input-side system 301 and transmits it to an output-side system 302.

For this, the digital therapy management system 300 may include a UX user measurement system 310 and a UX expert evaluation and analysis system 320.

The UX user measurement system 310 may collect implementation result information, such as a 3D depth camera, a webcam, a microphone, EEG, GSR, Interaction & Eye tracker, and a UX survey, obtained through multimodal sensors, and implement functions such as emotion recognition, cognitive recognition, interaction, automatic survey generation, and UX knowledge modeling.

In addition, these functions may be used to implement emotion metrics, cognitive metrics, interaction metrics, and automatic survey metrics.

In addition, the UX expert evaluation and analysis system 320 may calculate user evaluation indices (UXEI) such as attractiveness index, need index, and pleasure index using emotion metrics, cognitive metrics, interaction metrics, and automatic survey metrics.

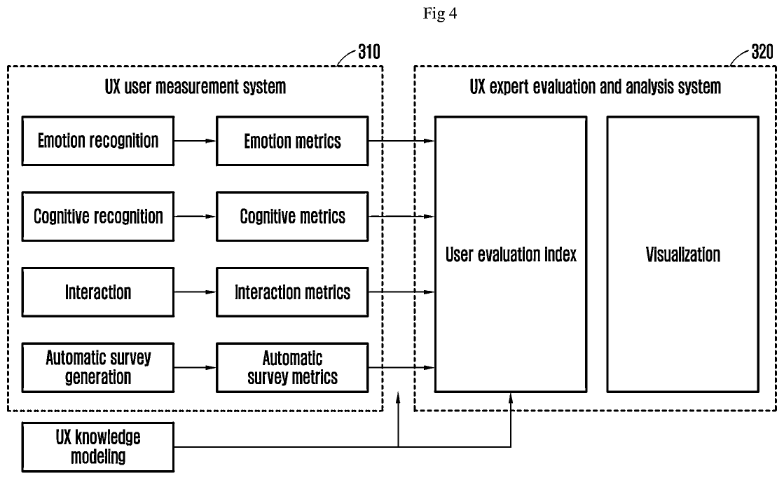

FIG. 4 is a drawing specifically explaining the UX user measurement system 310.

The UX user measurement system 310 may detect user's actions and gestures in real-time to analyze the interaction and measure the spatial characteristics.

In addition, user's facial expression and behavior may be captured and utilized for emotion recognition and interaction analysis, and user's motion and fatigue may be analyzed by detecting hand movement through an electromyography sensor.

In addition, EEG (brain waves) and GSR (skin conductance) may be measured to identify the user's emotional and cognitive state, analyze physical reactions, and track the user's eye movements and interactions to utilize them for cognitive and interaction analysis.

In addition, rich feedback may be obtained through a UX survey that collects subjective evaluations of user's experiences.

The UX user measurement system 310 may track and analyze the user's emotions and cognitive states in real-time through brainwaves, skin conductance, and facial expression analysis, analyze an interaction between the user and the system through a 3D depth camera and eye tracking, and seek ways to improve them.

Based on the variety of collected sensor data, an automated questionnaire about user experience may be generated to obtain deeper feedback, and a UX knowledge model may be built based on the collected data to understand patterns in user behavior and experience and may be applied to future designs and services.

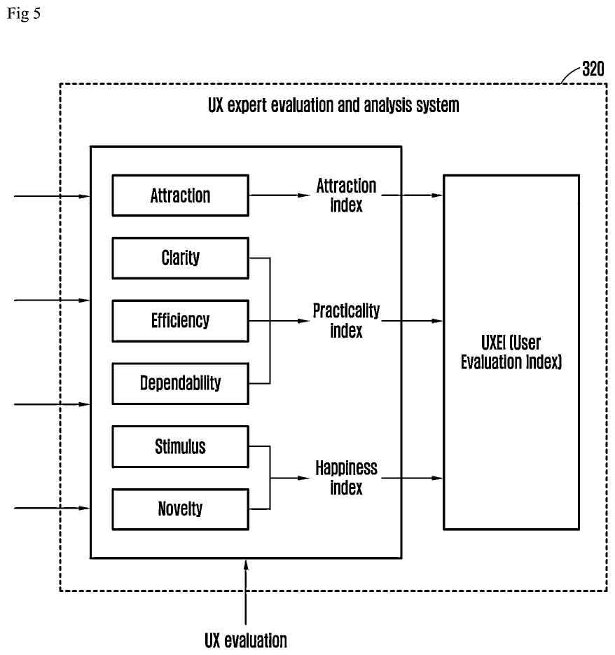

FIG. 5 specifically illustrates a UX expert evaluation and analysis system 500.

The UX expert evaluation and analysis system 500 may evaluate and analyze the user experience (UX) by utilizing various metrics. The main metrics and the user evaluation index (UXEI) may be calculated as follows.

Emotion metrics measure user's emotional states, and analyze user's emotions based on facial expressions, brain waves, and skin conductance data collected through webcams, EEG, GSR, etc.

Cognitive metrics measure the user's level of information processing and understanding, and use EEG and other brain activity measurement tools to evaluate the user's cognitive effort and cognitive load.

Interaction metrics measure the quality of interaction between users and systems, and may analyze the user's actions and gazes using 3D depth cameras, eye trackers, etc., thereby evaluating the effectiveness of the interaction.

Automatic survey metrics collect subjective evaluations by providing users with an automated survey about their experiences, and may provide users with automatically generated surveys based on various collected sensor data and interaction metrics to evaluate quality, satisfaction, inconvenience, etc.

The User Evaluation Index (UXEI) may be calculated through attractiveness index, practicality index, pleasure index, etc., and is calculated by synthesizing various metrics such as collected emotion metrics, cognitive metrics, interaction metrics, and automatic survey metrics.

The UX expert evaluation and analysis system may objectively and quantitatively evaluate and analyze the UX of a product, service, or system by comprehensively utilizing these metrics to suggest directions for improvement and contribute to user-centered design.

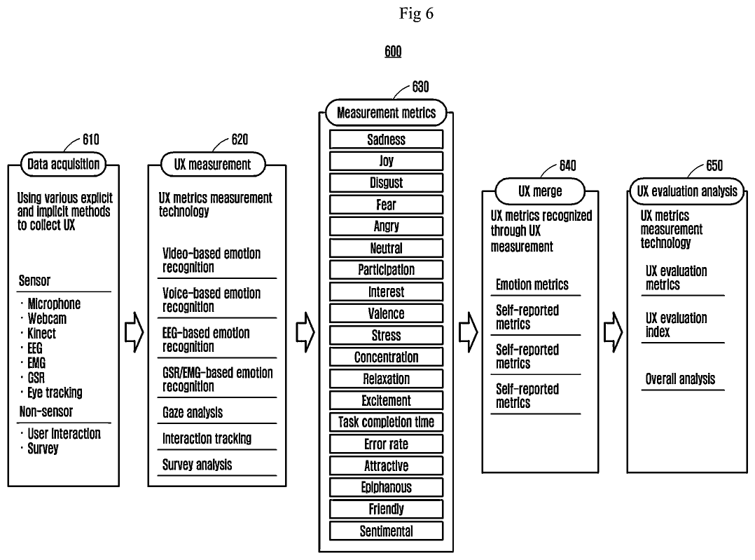

FIG. 6 illustrates a drawing 600 explaining UX merge and UX evaluation analysis through measurement metrics.

This UX measurement and evaluation platform may greatly improve the quality of DTx by helping DTx startups or small and medium-sized development companies that have difficulty hiring expensive UX experts to measure/evaluate the UX usability of their products.

In addition, the UX evaluation platform has the advantage of being able to conduct reliable analysis and secure objectivity through holistic UX measurement and analysis evaluation based on various multimodal sensor data.

UX measurement and evaluation results may be easily analyzed by UX experts through visualization. For reliable UX evaluation, this project develops a 10-step evaluation procedure which may include 1) identifying a set of UX evaluation methods, 2) identifying technologies, 3) prioritizing technologies, 4) identifying a usage environment, 5) selecting a subset of technologies, 6) preparing an evaluation environment, 7) applying UX evaluation technologies, 8) evaluating UX evaluation results, 9) reusing UX evaluation templates, and 10) using new technologies.

A data management module first goes through an asynchronous observation data acquisition step and a multimodal sensor data acquisition step, which requires synchronization, through the IoT/Cloud infrastructure. The acquired massive data is converted into metadata through the Packet Builder. The data and metadata created in this way may be classified into HDFS, EMR, and PHR through classification assumptions in a multidimensional storage space.

Based on the data finally created, a medical report may be created according to the query, and data required for the UX measurement and evaluation module and the DTx service may also be provided.

To implement the digital therapy management system according to the present disclosure, inter-hospital and existing hospitals are important to be interconnected, which may be supported by the HIS interconnection module.

EMR/EHR information obtained from a database and PHR data obtained from a medical device or wearable sensor are transmitted to an input adapter.

In the mapping application service, the knowledge-based extraction module, terminology maps, and Drizzle Data Archive may be obtained through the terminology standard code extractor, and the knowledge-based map may be created based on the normalized EMR handler and the knowledge-based semantic mapping generator, and then verified by medical staff.

The mapping management service consists of a schema storage, a matching module, and a mapping verification service module, and supports interoperability services with existing hospital information systems (HIS).

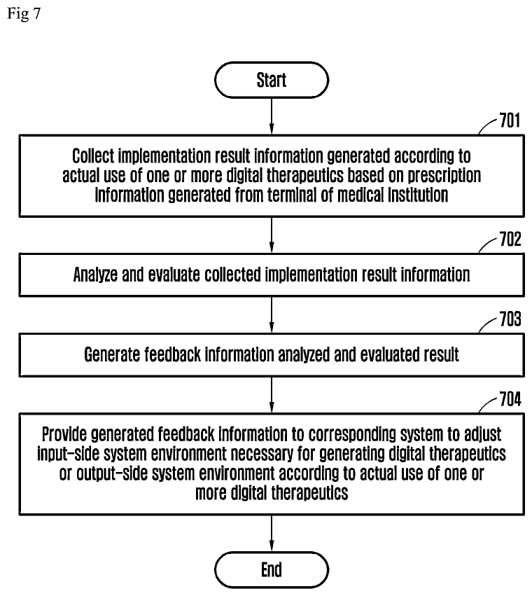

FIG. 7 is a drawing explaining an operation method of the digital therapy management system according to an embodiment.

The operation method of the digital therapy management system according to an embodiment may collect implementation result information generated according to the actual use of one or more digital therapeutics based on prescription information generated from a terminal of a medical institution (step 701).

In addition, the collected implementation result information may be analyzed and evaluated (step 702), and feedback information may be generated based on the analyzed and evaluated results (step 703).

Various technologies and methods may be utilized in the process of analyzing and evaluating the collected results information. Important trends and patterns may be identified by performing statistical analysis based on the collected data, and data characteristics may be understood and meaningful insights may be obtained through average, standard deviation, and frequency analysis.

Machine learning algorithms may be used to learn patterns from data and create predictive models, and, in particular, predictive models for individual patient responses may be used to develop specialized treatment methods or detect side effects in advance.

User's behavioral patterns and the effectiveness of the digital therapeutics may be analyzed in relation to each other. By understanding how specific behaviors affect the effectiveness, individual treatment methods may be optimized.

In the step of generating feedback information based on the analyzed and evaluated results, various forms of feedback may be developed. Based on the analysis results, customized feedback may be generated for each user. Feedback may be developed to optimize treatment methods by considering information about response patterns, performance, and areas for improvement of individual patients.

In addition, a report and recommendation on the effectiveness of specific digital therapeutics is generated for a medical professional by synthesizing the performance and characteristics of the entire patient population, which allows the medical professional to collectively optimize the treatment method or consider additional research and measures for a specific patient group.

Next, the operation method of the digital therapy management system according to an embodiment may provide the generated feedback information to the corresponding system to adjust the input-side system environment required to generate one or more digital therapeutics or the output-side system environment according to the actual use of the digital therapeutics (step 704).

To provide the generated feedback information to the corresponding system, feedback information for adjusting settings for at least one device among a multimodal sensor, VR/AR equipment, and wearable device for collecting user information may be provided as an input-side system environment necessary for generating one or more digital therapeutics.

In addition, to provide the generated feedback information to the corresponding system, feedback information that processes at least one data among questionnaire data, medical knowledge, or guidelines provided to the user may be provided as an input-side system environment necessary for generating one or more digital therapeutics.

To provide the generated feedback information to the corresponding system, the generated feedback information may be provided to the corresponding system to adjust the output-side system environment for a service provided by a physical hospital or virtual hospital that supports the actual use of one or more digital therapeutics.

In addition, to provide the generated feedback information to the corresponding system, the generated feedback information may be provided to the corresponding system to adjust the output side system environment that provides at least one service among digital literacy service, evidence-based/personalized service, or virtual hospital medical service as a service provided by a physical hospital or a virtual hospital.

Ultimately, by utilizing the present disclosure, it is possible to provide a user with optimized, high-quality digital therapeutics by linking various health information on the user with the medical records of a medical institution.

In addition, treatment results may be diagnosed more accurately based on user feedback on prescribed digital therapeutics, and users' medical records related to digital therapeutics may be more easily managed by users with just an update operation linked with an existing medical record database.

In accordance with an embodiment, the present disclosure can provide users with high-quality digital therapeutics optimized for them by linking their various health information with the medical records of a medical institution.

In accordance with an embodiment, the present disclosure can diagnose treatment outcomes more accurately based on user feedback on prescribed digital therapeutics.

In accordance with an embodiment, the present disclosure is linked to existing medical record database, making it easier to manage users' medical records related to digital therapeutics with just an update operation.

The apparatus described above may be implemented as a hardware component, a software component, and/or a combination of hardware components and software components. For example, the apparatus and components described in the embodiments may be achieved using one or more general purpose or special purpose computers, such as, for example, a processor, a controller, an arithmetic logic unit (ALU), a digital signal processor, a microcomputer, a field programmable array (FPA), a programmable logic unit (PLU), a microprocessor, or any other device capable of executing and responding to instructions. The processing device may execute an operating system (OS) and one or more software applications executing on the operating system. In addition, the processing device may access, store, manipulate, process, and generate data in response to execution of the software. For ease of understanding, the processing apparatus may be described as being used singly, but those skilled in the art will recognize that the processing apparatus may include a plurality of processing elements and/or a plurality of types of processing elements. For example, the processing apparatus may include a plurality of processors or one processor and one controller. Other processing configurations, such as a parallel processor, are also possible.

The software may include computer programs, code, instructions, or a combination of one or more of the foregoing, configure the processing apparatus to operate as desired, or command the processing apparatus, either independently or collectively. In order to be interpreted by a processing device or to provide instructions or data to a processing device, the software and/or data may be embodied permanently or temporarily in any type of a machine, a component, a physical device, a virtual device, a computer storage medium or device, or a transmission signal wave. The software may be distributed over a networked computer system and stored or executed in a distributed manner. The software and data may be stored in one or more computer-readable recording media.

The methods according to the embodiments of the present disclosure may be implemented in the form of a program command that can be executed through various computer means and recorded in a computer-readable medium. The computer-readable medium can store program commands, data files, data structures or combinations thereof. The program commands recorded in the medium may be specially designed and configured for the present disclosure or be known to those skilled in the field of computer software. Examples of a computer-readable recording medium include magnetic media such as hard disks, floppy disks and magnetic tapes, optical media such as CD-ROMs and DVDs, magneto-optical media such as floptical disks, or hardware devices such as ROMs, RAMs and flash memories, which are specially configured to store and execute program commands. Examples of the program commands include machine language code created by a compiler and high-level language code executable by a computer using an interpreter and the like. The hardware devices described above may be configured to operate as one or more software modules to perform the operations of the embodiments, and vice versa.

Although the present disclosure has been described with reference to limited embodiments and drawings, it should be understood by those skilled in the art that various changes and modifications may be made therein. For example, the described techniques may be performed in a different order than the described methods, and/or components of the described systems, structures, devices, circuits, etc., may be combined in a manner that is different from the described method, or appropriate results may be achieved even if replaced by other components or equivalents.

Therefore, other embodiments, other examples, and equivalents to the claims are within the scope of the following claims.

What is claimed is:

1. A digital therapy management system, comprising:
an implementation result manager for collecting implementation result information generated according to actual use of one or more digital therapeutics based on prescription information generated from a terminal of a medical institution;
an analyzer/evaluator for analyzing and evaluating the collected implementation result information;
a feedback information generator for generating feedback information based on the analyzed and evaluated result; and
a feedback information provider for providing the generated feedback information to a corresponding system to adjust an input-side system environment required to generate the digital therapeutics or an output-side system environment according to the actual use of the digital therapeutics.

2. The digital therapy management system according to claim 1, wherein the feedback information provider provides feedback information for adjusting settings for at least one device among a multimodal sensor, VR/AR equipment, or wearable device for collecting user information as an input-side system environment necessary for generating the digital therapeutics.

3. The digital therapy management system according to claim 1, wherein the feedback information provider provides feedback information processing one or more data of questionnaire data, medical knowledge, and guidelines provided to a user as an input-side system environment necessary for generating the digital therapeutics.

4. The digital therapy management system according to claim 1, wherein the feedback information provider provides the generated feedback information to a corresponding system to adjust an output-side system environment for a service provided by a physical hospital or virtual hospital that supports the actual use of the digital therapeutics.

5. The digital therapy management system according to claim 4, wherein the feedback information provider provides the generated feedback information to a corresponding system so as to control the output-side system environment that provides one or more services among digital literacy service, evidence-based/personalized service, and virtual hospital medical service as a service provided by a physical hospital or a virtual hospital.

6. The digital therapy management system according to claim 1, wherein the implementation result manager collects at least one of data, input by a user, and bio-signal data of the user as the implementation result information in response to the provided prescription information,
the data input by the user comprises at least one of the number of times of implementation, implementation time, and implementation intensity of digital therapeutics corresponding to the prescription information, and
the bio-signal data of the user is monitored through at least one of a smart terminal and IoT terminal of the user and comprises at least one of blood pressure, blood sugar, body temperature, heart rate, moving distance, and moving steps of the user.

7. The digital therapy management system according to claim 1, wherein the implementation result manager provides the prescription information to a user's terminal comprising a multimodal sensor, receives implementation result information corresponding to the provided prescription information from the user's terminal, and provides the received implementation result information to the terminal of the medical institution.

8. The digital therapy management system according to claim 1, further comprising a record manager configured to receive treatment result information corresponding to the implementation result information from the terminal of the medical institution and to update the received treatment result information in electronic medical record (EMR) database of the medical institution,
wherein the treatment result information comprises at least one information of the prescription information, the implementation record information, medical professional's comment information according to the implementation record information and the medical professional's authentication information.

9. An operation method of a digital therapy management system, the operation method comprising:
collecting implementation result information generated according to actual use of one or more digital therapeutics based on prescription information generated from a terminal of a medical institution;
analyzing and evaluating the collected implementation result information;
generating feedback information based on the analyzed and evaluated result; and
providing the generated feedback information to a corresponding system to adjust an input-side system environment required to generate the digital therapeutics or an output-side system environment according to the actual use of the digital therapeutics.

10. The operation method according to claim 9, wherein the providing comprises providing feedback information for adjusting settings for at least one device among a multimodal sensor, VR/AR equipment, or wearable device for collecting user information as an input-side system environment necessary for generating the digital therapeutics.

11. The operation method according to claim 9, wherein the providing comprises providing feedback information processing one or more data of questionnaire data, medical knowledge, and guidelines provided to a user as an input-side system environment necessary for generating the digital therapeutics.

12. The operation method according to claim 9, wherein the providing comprises providing the generated feedback information to a corresponding system to adjust an output-side system environment for a service provided by a physical hospital or virtual hospital that supports the actual use of the digital therapeutics.

13. The operation method according to claim 12, wherein the providing comprises providing the generated feedback information to a corresponding system so as to control the output-side system environment that provides one or more services among digital literacy service, evidence-based/personalized service, and virtual hospital medical service as a service provided by a physical hospital or a virtual hospital.
